(19) United States
(12) Patent Application Publication
(10) Pub. No.: US 2013/0204136 A1
(43) Pub. Date: Aug. 8, 2013

(54) SYSTEM AND METHOD FOR IMAGING A VOLUME OF TISSUE

(71) Applicant: Delphinus Medical Technologies, Inc., Plymourth, MI (US)

(72) Inventors: Nebojsa Duric, Bloomfield Hills, MI (US); Olivier Roy, Royal Oak, MI (US); Steven Schmidt, Clinton Twp, MI (US); Cuiping Li, Troy, MI (US); Roman Janer, Plymouth, MI (US); Xiaoyang Cheng, Ann Arbor, MI (US); Jefferey Goll, Lake Oswego, OR (US)

(73) Assignee: Delphinus Medical Technologies, Inc., Plymourth, MI (US)

(21) Appl. No.: 13/756,851

(22) Filed: Feb. 1, 2013

Related U.S. Application Data
(60) Provisional application No. 61/643,431, filed on May 7, 2012.
(60) Provisional application No. 61/594,877, filed on Feb. 3, 2012.

(51) Int. Cl. A61B 8/00; A61B 8/08; A61B 8/15

(52) U.S. Cl. CPC A61B 8/4488; A61B 8/15; A61B 8/54; A61B 8/406; A61B 8/5207; A61B 8/5223; A61B 8/461; A61B 8/467
USPC 600/448

(57) ABSTRACT

A system and method for imaging a volume of tissue comprising: a modular transducer array, configured to substantially surround the volume of tissue, emit acoustic waveforms toward the volume of tissue, and receive acoustic waveforms scattered by the volume of tissue, comprising a first and a second modular transducer subarray configured to couple to one another; a controller configured to control acoustic signals emitted by the first and the second modular transducer subarrays; an electronic subsystem, coupled to the modular transducer array, comprising a multiplexor and beam-forming elements and configured to receive a set of acoustic data from the first and the second modular transducer subarrays; and a processor configured to analyze the set of acoustic data, determine the distribution of at least one acoustomechanical parameter within the volume of tissue, and render an image of the volume of tissue based on the acoustomechanical parameter.

### CROSS-REFERENCE TO RELATED APPLICATIONS

This application claims the benefit of U.S. Provisional Application Ser. No. 61/594,877, filed on 3 Feb. 2012 and U.S. Provisional Application Ser. No. 61/643,431, filed on 7 May 2012, which are incorporated in their entirety by this reference.

### TECHNICAL FIELD

This invention relates generally to the medical imaging field, and more specifically to an improved system and method for imaging a volume of tissue.

### BACKGROUND

Early detection and treatment of breast cancer and other kinds of cancer typically result in a higher survival rate. Despite a widely accepted standard of mammography screenings for breast cancer detection, there are many reasons that cancer is often not detected early. One reason is low participation in breast screening, as a result of limited access to equipment and fear of radiation and discomfort. Another reason is limited performance of mammography, particularly among women with dense breast tissue, who are at the highest risk for developing breast cancer. As a result, many cancers are missed at their earliest stages when they are the most treatable. Furthermore, mammography results in a high rate of “false alarms”, leading to unnecessary biopsies that are collectively expensive and result in emotional duress in patients.

Other imaging technologies in development are unlikely to create a paradigm shift towards early detection of cancer. For example, magnetic resonance (MR) imaging can improve on some of these limitations by virtue of its volumetric, radiation-free imaging capability, but requires long exam times and use of contrast agents. Furthermore, MR has long been prohibitively expensive for routine use. As another example, positron emission tomography is also limited by cost. Conventional sonography, which is inexpensive, comfortable and radiation-free, is not a practical alternative because of its operator dependence and the long time needed to scan the whole breast. In other words, lack of a low-cost, efficient, radiation-free, and accessible tissue imaging alternative to mammography is a barrier to dramatically impacting mortality and morbidity through improved screening because, currently, there is a trade-off between the cost effectiveness of mammography and the imaging performance of MR.

Thus, there is a need in the medical imaging field to create an improved system and method for imaging a volume of tissue that addresses the need to combine the low-cost advantage of mammography with superior imaging performance. This invention provides such an improved system and method.

### BRIEF DESCRIPTION OF THE FIGURES

FIGS. 1A and 1B are schematics of the system of a preferred embodiment;

FIGS. 2A and 2B are schematics of the transducer array in the system of a preferred embodiment;

FIGS. 3A and 3B are schematics of the transducer subarrays and electronic subsystem in the system of a preferred embodiment;

FIGS. 4 and 5 are flowcharts depicting the method of a preferred embodiment; and

FIGS. 6A-6D depict an illustrative exemplary preferred embodiment of the system and method.

### DESCRIPTION OF THE PREFERRED EMBODIMENTS

The following description of preferred embodiments of the invention is not intended to limit the invention to these preferred embodiments, but rather to enable any person skilled in the art to make and use this invention.

### System for Imaging a Volume of Tissue

As shown in FIGURES IA, 1B, 2A, 2B, 3A, AND 3C, system 100 of a preferred embodiment includes: a transducer array 110 configured to substantially surround the volume of tissue 102, including a plurality of modular transducer subarrays 112 coupled to one another and each including a series of emitters 114 for irradiating the volume of tissue 102 with acoustic signals 118 and a series of detectors 116 for receiving the acoustic signals scattered by the volume of tissue 102; an electronic subsystem 130 coupled to the transducer array no and configured to receive acoustic data from each transducer subarray; and a processor 140 configured to analyze the received acoustic data and generate an image rendering of the volume of the tissue 102 based on at least one acoustic parameter.

The preferred system 100 provides a non-ionizing and safe imaging modality that is low-cost and produces high-resolution images of tissue 102 within a relatively brief period of time. Furthermore, the preferred system 100 is modular, and preferably scalable to accommodate continual improvements in computing and/or electronic efficiency. In particular, the imaging efficiency of the preferred system can correlate with Moore's law, which is a rule of thumb regarding the exponential rate of improvements in, for example, processing speed in electronic devices. The modularity and scalability preferably enables retrofitting of outdated versions of the preferred system, which increases longevity of the preferred system and reduces long-term costs, thereby improving accessibility of the preferred system for cancer screening purposes. The system is preferably used to image breast tissue, but can additionally or alternatively be used to image any suitable kind of tissue.

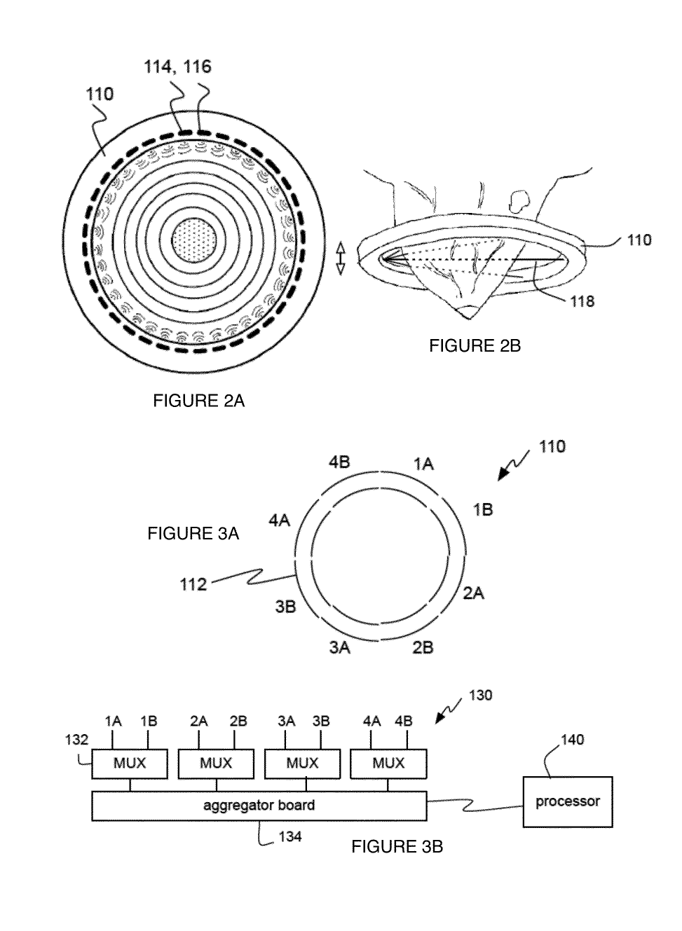

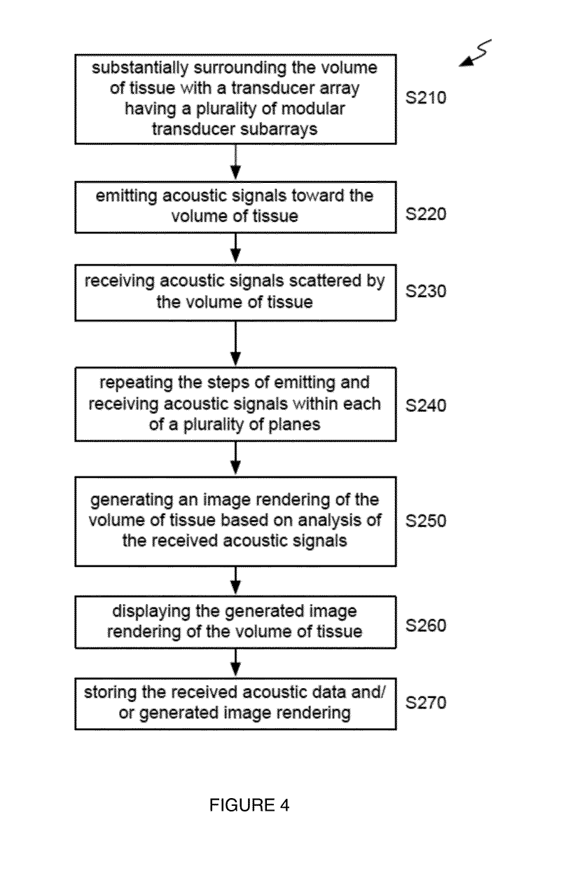

As shown in FIGS. 2A and 2B, the transducer array no preferably functions to transmit and receive acoustic signals 118 to generate acoustic data regarding the interactions between acoustic signals 118 and the volume of tissue 102. As shown in FIG. 3A, the transducer preferably includes a plurality of modular transducer subarrays 112 coupleable to one another such as to collectively and substantially surround the volume of tissue 102. Each transducer subarray preferably includes a series of ultrasound emitters 114 configured to emit acoustic signals 118 towards the volume of tissue 102 and a series of ultrasound detectors 116 configured to receive acoustic signals 118 scattered by the volume of tissue 102. In one preferred embodiment, the transducer array 110 can include one or more instances of a single physical element that includes a set of transmitting and receiving detectors, and that can be controlled by a switch or other suitable controlling feature to selectively operate in either the transmitting or receiving/detecting mode. The emitted acoustic signals 118 preferably interact with the volume of tissue 102 (or other irradiated object) according to acousto-mechanical properties of the tissue, and the received acoustic signals 118 can be analyzed to provide measurements of these acousto-mechanical properties of the tissue. In particular, the received acoustic signals 118 can be analyzed to provide measurements of acoustic reflectivity (based on the reflection of acoustic waves from the tissue), acoustic attenuation (based on amplitude changes of acoustic waves in the tissue), acoustic speed (based on departure and arrival times of acoustic signals 118 between emitter-receiver pairs), and/or any suitable acoustic parameter, such as elasticity.

The plurality of transducer subarrays 112 preferably couple to one another to form the transducer array no. The transducer subarrays 112 preferably couple to a transducer frame that aligns and couples the transducer subarrays 112 to one another, and/or the transducer subarrays 112 can couple directly to one another, such as by interlocking with mating features. As shown in FIG. 2A, in one preferred embodiment, the transducer array 110 is a ring, of either substantially elliptical or substantially circular dimensions, and each transducer subarray includes an arc segment of the ring. The transducer subarrays 112 are preferably substantially identical such that the transducer array no is radially symmetrical, and the transducer subarrays 112 have approximately equal arc lengths. However, alternatively the transducer array no can include transducers subarrays 112 that are of different shapes and sizes, and the transducer array 110 can be of any suitable shape. The transducer subarrays 112 are preferably configured to be removable for swapping in and out of the transducer ring. For example, a first transducer subarray 112 having a first number of active elements (emitters and receivers) can be swapped with a second transducer subarray 112 having a second number of active elements different from the first transducer subarray 112. As technology continually improves and computing power increases (e.g., according to Moore's law), transducer subarrays 112 with more active elements can be swapped in a modular fashion into the preferred system 100, thereby enabling higher image resolution without requiring complete replacement of the entire system. Furthermore, transducer subarrays 112 with more closely spaced active elements can be swapped in a modular fashion to reduce the level of artifacts in the final image representation or representations of the volume of tissue 102. In another example, the transducer subarrays 112 can be reconfigured in different arrangements to accommodate different sizes and/or shapes of tissue, or to accommodate any suitable object other than tissue. One modular aspect of the transducer array 110 preferably reduces long-term costs of maintaining cutting-edge imaging capabilities, thereby increasing accessibility of highly accurate, beneficial imaging screening technology. Another modular aspect of the transducer array 110 can be achieved at a higher level, with multiple transducer arrays no in combination. For example, two or more transducer arrays no can be stacked vertically or combined in any suitable arrangement to form a two-dimensional surface. Various combinations of multiple transducer arrays no can create different surfaces, such as that resulting from stacked ring-shaped transducer arrays no, stacked arc segment-shaped transducer arrays no, or any combination thereof. In other words, the transducer array 110 can provide modularity using sub-arrays within the transducer array 110, and/or modularity at a higher level using multiple transducer arrays no.

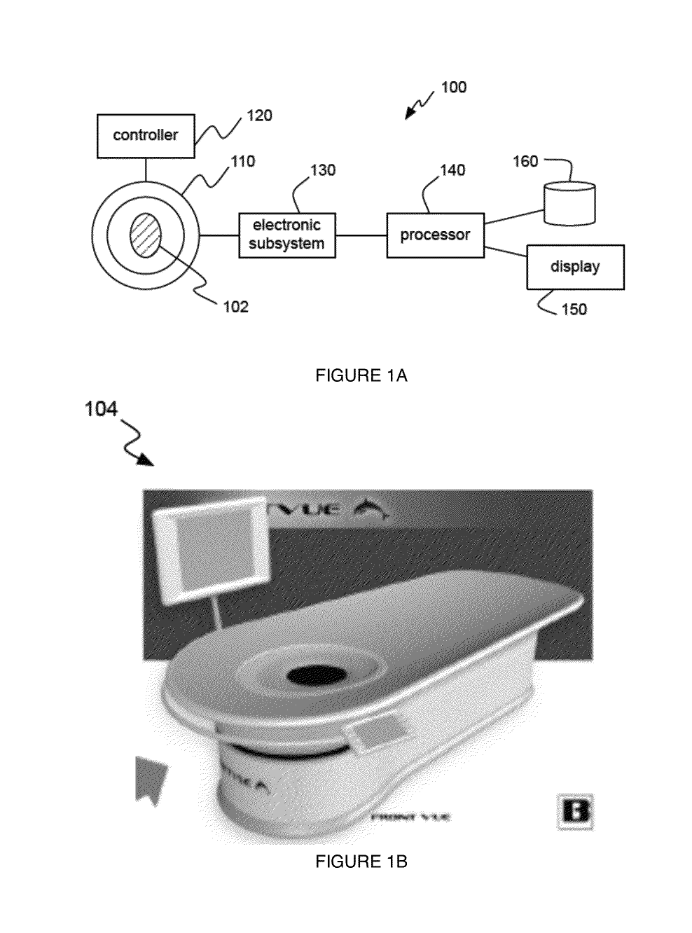

As shown in FIG. 1B, in a preferred embodiment of the system 100, the transducer ring can be paired with a patient table 104 having an aperture, such that a patient lying prone stomach-side down on the patient table can pass her breast through the aperture. The patient table is preferably set up with a water bath, positioned beneath the patient table aperture, that receives the breast tissue and houses the transducer array 110 of the preferred system. The transducer array no, while surrounding the breast, preferably moves sequentially to a series of points along a vertical path, scanning a two-dimensional cross-sectional image (e.g., coronal image) of the breast at each point, such that the processor 140 can use the received acoustic data to form a stack or series of two-dimensional images over the entire volume of tissue (and/or a three-dimensional image of the volume of tissue). The water bath preferably functions to act as an acoustic coupling medium between the transducer array 110 and the tissue, and to suspend the breast tissue (thereby reducing gravitational distortion of the tissue). In this embodiment, the preferred system 100 can include a controller 120 that functions to control the actions of the transducer array 110. The controller 120 preferably functions to control the acoustic signals 118 transmitted from the ultrasound emitters 114. In particular, the controller 120 preferably controls the frequency of emitted acoustic signals 118, and/or the frequency of activation of the ultrasound emitters 114. The controller 120 preferably further controls the physical movements of the transducer array no relative to the volume of tissue. In particular, the controller 120 preferably controls motion of the transducer array no, including dictating spacing between the scanning points at which the scanning occurs and rate of travel between the scanning points. In a first variation, the controller 120 may stop the ring at specific points before scanning sequential slices of the volume of tissue; however, in another variation, the controller may be configured to allow for continuous scanning of the volume of tissue. In alternative embodiments, the system 100 can be paired with any suitable patient setup that allows the transducer array no to substantially surround the volume of tissue to be scanned.

The electronic subsystem 130 preferably functions to receive acoustic data from the transducer subarray. As shown in FIG. 3B, the electronic subsystem 130 preferably includes a plurality of multiplexers 132. Each multiplexer 132 is preferably coupled to at least two transducer subarrays 112, and selects in turn each of the transducer subarrays 112 and forwards the signal from the selected transducer subarray to an aggregator board 134 that collects the multiplexed signals. The signals from the aggregator board 134 are then forwarded onto the processor 140 for analysis and reconstruction into an image rendering of the volume of tissue 102. The multiplexers 132 are preferably electronic multiplexers 132, but can additionally or alternatively include analog or mechanical multiplexers (e.g. for controlling the up and down motion of the preferred embodiment) 132. The electronic subsystem 130 can further include any suitable signal processing components, such as analog-digital converters, transmit and receive beam-formers, and/or amplifiers.

Similar to the transducer array no, at least the multiplexers 132 are preferably modular such that the multiplexers 132 can be swapped and replaced with other multiplexers 132. For example, an embodiment of the preferred system 100 having a series of 2:1 multiplexers (two input channels and one output channel) is preferably capable of being modified to instead include a series of 3:1 multiplexers (three input channels and one output channel) or other suitable kind of multiplexers, such as to accommodate an updated version of the transducer array 110 having more active elements. The use of modular multiplexers 132 thus provides a tradeoff between system complexity and acquisition time (i.e. multiplexing reduces the number of acquisition channels required, but increases the acquisition time). The system 100 thus preferably uses an optimized amount of multiplexing, governed by the multiplexers 132, such that a ratio of multiplexing to acquisition time is optimized for an application. Alternatively, the system 100 may not use an optimized amount of multiplexing.

The processor 140 preferably functions to generate an image rendering of the volume of tissue 102 based on at least one acoustic parameter determined from the received acoustic data. The processor 140 preferably includes a reconstruction engine that performs acoustic tomography, a technique that uses computed tomography methods to solve an inverse problem involving acoustic signals 118. Using acoustic tomographic methods, the processor 140 preferably infers acousto-mechanical properties of the scanned volume of tissue 102 from the received acoustic data measured by the transducer array 110 along a surface surrounding the tissue 102. The processor 140 can implement any suitable tomographic method. For example, the processor 140 can implement bent ray tomography, beamforming or SAT techniques for reflection imaging, straight ray tomography (backprojection) for transmission imaging, curved ray tomography, and/or waveform tomography, versions of which are known and readily understood by one ordinarily skilled in the art.

In one embodiment, the processor 140 of the preferred system 100 generates a “stack” of two-dimensional images representing a series of cross-sections of the volume of tissue 102. The processor 140 can additionally or alternatively generate a three-dimensional volumetric rendering based on the stack of two-dimensional images, and/or generate a three-dimensional volumetric rendering directly based on the received acoustic data. An image representation of any portion of the volume of tissue 102 can depict any one or more acousto-mechanical properties of the volume of tissue 102. For example, an image representation can depict acoustic attenuation, acoustic reflection, acoustic speed, and/or any suitable property of the tissue 102. As described further in U.S. Patent Application Publication No. U.S. 2011/0201932, the entirety of which is incorporated herein by this reference, any combination of acousto-mechanical properties of the tissue 102 can be combined in a particular single image rendering based on thresholds for each property, or in any suitable manner.

The processor 140 is preferably implemented on a blade server or other suitable modular computer system 100. Similar to the transducer array, the processor 140 (and/or other computing elements) is preferably modular such that as technological capabilities are expanded over time, the processor 140 and/or other computing elements can be swapped and replaced with updated, preferably more efficient versions of the processor 140 and/or other computing elements. In alternative embodiments, the processor 140 is implemented in any suitable computing process, such as cluster computing or cloud computing. In the preferred embodiment, the blade server contains 8 or more computing blades, each of which preferably contains multiple CPUs and GPUs. An aspect of modularity is therefore preferably achieved at the blade level (e.g., using multiple blades), and another aspect of modularity is also preferably achieved within the level of each blade (e.g., using the multiple CPU and GPU components within each blade).

In a preferred embodiment, the system 100 further includes a display 150 configured for displaying one or more of the generated image renderings of the tissue 102, such as on a computer or other user interface to a medical technician, physician, or other medical practitioner. The preferred system 100 can additionally or alternatively include a server 160 or other storage device for storing the received acoustic data and/or generated image renderings. The preferred system 100 can additionally or alternatively be configured to store the data and/or generated image renderings in an electronic medical record or other storage associated with a patient being scanned.

### Method for Imaging a Volume of Tissue

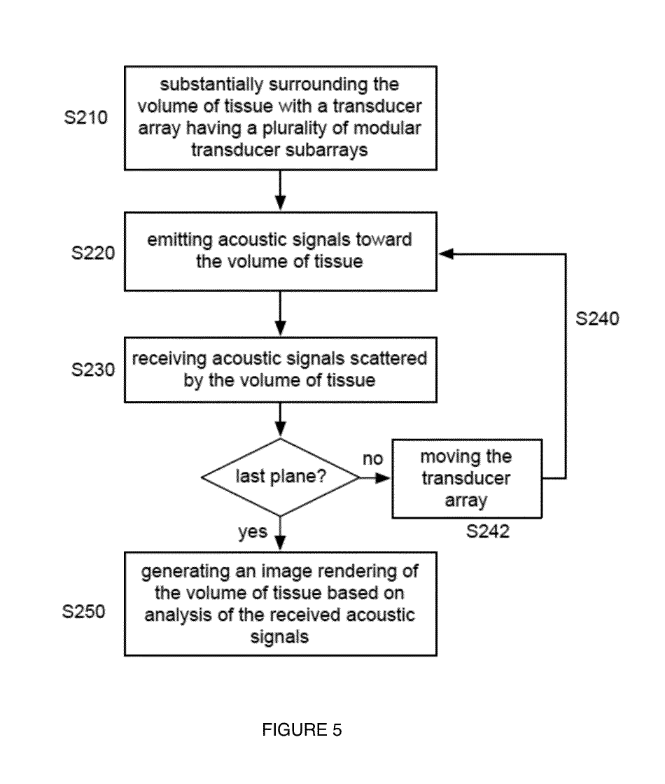

As shown in FIG. 4, a method 200 of the preferred embodiment includes: in block S210, substantially surrounding the volume of tissue with a transducer array having a plurality of modular transducer subarrays; in block S220, emitting acoustic signals toward the volume of tissue; in block S230, receiving acoustic signals scattered by the volume of tissue; in block S240, repeating the steps of emitting and receiving acoustic signals within each of a plurality of planes, each plane located at a respective point along an axis of the volume of tissue; and in block S250, generating an image rendering of the volume of tissue based on analysis of the received acoustic signals.

The method 200 is preferably used to image breast tissue, but can additionally or alternatively be used to image any suitable kind of tissue. The preferred method provides a non-ionizing and safe imaging modality that is low-cost and produces high-resolution images of tissue within a relatively brief period of time. Furthermore, in one preferred embodiment, the method is used with a modular and scalable ultrasound scanning system to accommodate continual improvements in computing efficiency and/or other desired changes.

As shown in FIG. 4, block S210 of the preferred method includes substantially surrounding the volume of tissue with a transducer array having a plurality of modular transducer subarrays. Block S210 preferably functions to position the transducer array relative to the object to be scanned. Each transducer subarray preferably includes ultrasound emitters and ultrasound receivers, which can be combined in a plurality of transceivers or distributed as separate emitter and receiver elements. In block S210, the transducer array preferably surrounds the volume of tissue within a plane, and emitters and receivers are preferably distributed approximately uniformly around the tissue. The transducer array preferably includes a plurality of modular transducer subarrays that can be swapped for other transducer subarrays and/or reconfigured in other arrangements. In one preferred embodiment, the transducer array is a circular transducer ring having modular transducer subarrays that are of approximately equal arc length.

As shown in FIG. 4, block S220 of the preferred method recites emitting acoustic signals toward the volume of tissue, and block S230 recites receiving acoustic signals scattered by the volume of tissue. Blocks S220 and S230 preferably function to scan a cross-sectional image of the tissue using acoustic signals and to obtain data regarding acousto-mechanical properties of the tissue. The received acoustic signals can be analyzed to provide measurements of acoustic reflectivity (based on the reflection of acoustic waves from the tissue), acoustic attenuation (based on amplitude changes of acoustic waves in the tissue), acoustic speed (based on departure and arrival times of acoustic signals between emitter-receiver pairs), and/or any suitable acoustic parameter. The acoustic signals can be emitted from around the transducer array approximately simultaneously, or can be emitted in a sequential fashion around the transducer array.

As shown in FIG, 4, block S240 of the preferred method recites repeating the steps of emitting and receiving acoustic signals within each of a plurality of planes, each plane located at a respective point along an axis of the volume of tissue. Block S240 preferably functions to scan multiple cross-sectional images of the tissue using acoustic data. This repeating process is alternatively depicted in the flowchart of FIG. 5, where block S240 is represented by arrow S240. In a preferred embodiment of the method, blocks S220 and S230 are first performed within a first plane normal to a point located at the end of an axis of the volume of tissue. In this embodiment, the preferred method further includes moving the transducer array from the first plane to a second plane normal to a second point along the axis of the volume of tissue in block S242, and then performing blocks S220 and S230 of emitting and receiving acoustic signals within the second plane. The distance moved between the first and second planes can depend on, for example, the length of the tissue volume, desired spatial resolution of the resulting image of the tissue, or desired total scan time. Blocks S220 and S230 of emitting and receiving acoustic signals are preferably additionally repeated at any suitable number of planes normal to the axis. In one preferred embodiment of the method for scanning breast tissue, the transducer moves from a posterior portion of the tissue (i.e. from the chest wall region of a volume of breast tissue) to an anterior portion of the tissue (i.e. toward the tipple region of a volume of breast tissue). In another embodiment of the method for scanning breast tissue, the transducer moves from an anterior portion of the tissue (i.e., the nipple region of the breast) to a posterior portion of the tissue (i.e., toward the axilla region). However, the transducer array can be moved along any suitable axis of the tissue.

As shown in FIG. 4, block S250 of the preferred method recites generating an image rendering of the volume of tissue based on analysis of the received acoustic signals. The image rendering is preferably based on at least one acoustic parameter determined from the received acoustic signals. Block S250 preferably includes performing acoustic tomography, a technique that uses computed tomography methods to solve an inverse problem involving acoustic signals. Using acoustic tomographic methods, block S250 preferably infers acousto-mechanical properties of the scanned volume of tissue from the received acoustic data measured by the transducer array along a surface surrounding the tissue. Alternatively or additionally, block S250 can implement any suitable tomographic method. For example, the block S250 can implement bent ray tomography, beamforming or SAT techniques for reflection imaging, straight ray tomography (backprojection) for transmission imaging, curved ray tomography, and/or waveform tomography, versions of which are known and readily understood by one ordinarily skilled in the art.

Block S250 preferably generates a “stack” of two-dimensional images representing a series of cross-sections of the volume of tissue. Block S250 can additionally or alternatively generate a three-dimensional volumetric rendering based on the stack of two-dimensional images, and/or generate a three-dimensional volumetric rendering directly based on the received acoustic data. An image representation of any portion of the volume of tissue can depict any one or more acousto-mechanical properties of the volume of tissue. For example, an image representation can depict acoustic attenuation, acoustic reflection, acoustic speed, and/or any suitable property of the tissue. As described further in U.S. Patent Application Publication No. U.S. 2011/0201932, the entirety of which is incorporated herein by this reference, any combination of acousto-mechanical properties of the tissue can be combined in a particular single image rendering based on thresholds for each property, or in any suitable manner.

As shown in FIG. 4, the preferred method 200 further includes displaying the generated image rendering of the volume of tissue in block S260, such as on a computer or other user interface to a medical technician, physician, or other medical practitioner. The preferred method can additionally or alternatively include storing the received acoustic data and/or generated image renderings in block S270 on a server or other storage device. The preferred method can additionally or alternatively including the data and/or generated image renderings in an electronic medical record or other storage associated with a patient being scanned.

Variations of the preferred method include every combination and permutation of the processes described above. Furthermore, the system and method of the preferred embodiment can be embodied and/or implemented at least in part as a machine configured to receive a computer-readable medium storing computer-readable instructions. The instructions are preferably executed by computer-executable components preferably integrated with the system and one or more portions of the processor 140 and/or the controller 120. The computer-readable medium can be stored on any suitable computer-readable medium such as RAMs, ROMs, flash memory, EEPROMs, optical devices (CD or DVD), hard drives, floppy drives, or any suitable device. The computer-executable component is preferably a general or application specific processor, but any suitable dedicated hardware device or hardware/firmware combination device can alternatively or additionally execute the instructions.

### EXAMPLE

The following example implementation of the preferred system and method are for illustrative purposes only, and should not be construed as definitive or limiting of the scope of the claimed invention.

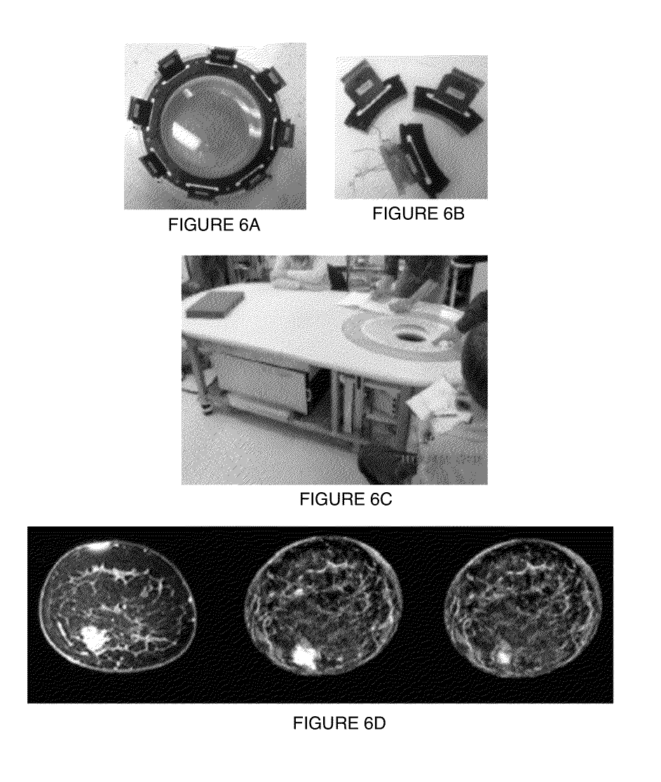

As shown in FIGS. 6A and 6B, in one illustrative embodiment, the example system includes an ultrasound transducer array in the shape of a twenty-centimeter diameter circular ring and having eight modular transducer subarrays of approximately equal arc length. Each transducer subarray includes two hundred fifty six active elements (transceivers), for a total of two thousand forty eight elements. The example system further includes a controller that drives the transducer array to emit ultrasound signals at an operating frequency of 2.75 MHz, and moves the transducer array between a plurality of imaging planes or “slices” along an axis. Given this operating frequency, the transducer elements are preferably spaced approximately half a signal wavelength apart.

As shown in FIG. 6C, during a scan, the ring transducer is submerged in a tank including between approximately two and one half and five gallons of water. The ring transducer receives in its center a volume of breast tissue to be scanned. At a beginning plane at a posterior end of the breast (highest imaging plane, in reference to a gravitational frame), the ring transducer emits ultrasound pulses toward the tissue and receives ultrasound pulses scattered by interaction with the tissue. After an approximately 30 ms scan period, the controller moves sequentially through the plurality of imaging planes separated by approximately one and one half millimeters, towards an end plane at a anterior end of the breast (lowest imaging plane, in reference to a gravitational frame). Scan of the entire breast takes approximately one minute or less.

The example system includes four 2:1 multiplexers that selectively forward the received signals to an aggregator board. The received acoustic data has a resolution of fourteen bits acquired at a rate of approximately five GB/second. The acoustic data is received by the processor, implemented in a modular blade computer, that generates a separate image rendering of the tissue based on each of acoustic reflection, acoustic speed, and acoustic attenuation similar to those shown in FIG. 6D. With an operating frequency of 2.75 MHz, the spatial resolution of the image rendering is approximately 1 mm, with an in-plane image pixel size of approximately 0.5 mm for acoustic reflection images, and approximately one millimeter for acoustic speed and acoustic attenuation. Compared to conventional ultrasound tomography technology, the resulting image renderings also have suppressed artifacts.

As a person skilled in the art will recognize from the previous detailed description and from the figures and claims, modifications and changes can be made to the preferred embodiments of the invention without departing from the scope of this invention defined in the following claims.

What is claimed is:

1. A system for imaging a volume of tissue comprising:
a transducer array, configured to substantially surround the volume of tissue, comprising a first modular transducer subarray and a second modular transducer subarray,
wherein the first modular transducer subarray is configured to couple to the second modular transducer subarray, and
wherein the first and second modular transducer subarrays each comprise a series of ultrasound emitters configured to emit acoustic waveforms toward the volume of tissue, and a series of ultrasound receivers configured to receive acoustic waveforms scattered by the volume of tissue;
a controller configured to control acoustic signals emitted by the first and second modular transducer subarrays;
an electronic subsystem, coupled to the transducer array, comprising a multiplexor configured to receive a set of acoustic data from the first and second modular transducer subarrays; and
a processor configured to analyze the set of acoustic data and render an image of the volume of tissue based on an acoustic parameter.

2. The system of claim 1, wherein the multiplexor is configured to select one of the first and second modular transducer subarrays and forward a signal from a selected modular transducer subarray to an aggregator board.

3. The system of claim 1, wherein the processor comprises a reconstruction engine configured to perform acoustic tomography.

4. The system of claim 3, wherein the processor is configured to implement at least one of bent ray tomography, beamforming techniques, and scanning acoustic tomography.

5. The system of claim 1, wherein the processor is implemented on a blade server.

6. The system of claim 1, wherein the transducer array further comprises a third modular transducer subarray, a fourth modular transducer subarray, and a second multiplexor configured to receive a second set of acoustic data from the third and fourth modular transducer subarrays.

7. The system of claim 6, wherein one of the first and second modular transducer subarrays is configured to couple to one of the third and fourth modular transducer subarrays.

8. The system of claim 1, wherein the multiplexor comprises two input channels and one output channel.

9. The system of claim 1 wherein the multiplexor is an electronic multiplexor.

10. The system of claim 1, wherein the controller controls at least one of a frequency of emitted acoustic signals and a frequency of activation of an ultrasound emitter.

11. A method for imaging a volume of tissue comprising:
substantially surrounding the volume of tissue with a transducer array comprising a first modular transducer subarray and a second modular transducer subarray;
emitting acoustic signals toward the volume of tissue and receiving acoustic signals scattered by the volume of tissue within each of a series of planes;
generating a set of acoustic data based on acoustic signals scattered by the volume of tissue and received by an electronics system comprising a multiplexor;
determining a distribution of a first acoustomechanical parameter, within the volume of tissue, based on the set of acoustic data; and
rendering an image of the volume of tissue based on the distribution of the first acoustomechanical parameter.

12. The method of claim 11, wherein determining a distribution of the first acoustomechanical parameter comprises performing acoustic tomography.

13. The method of claim 11, wherein determining a distribution of the first acoustomechanical parameter comprises determining a distribution of one of acoustic reflection, acoustic attenuation, and acoustic speed within the volume of tissue.

14. The method of claim 13, further comprising determining a distribution of a second acoustomechanical parameter within the volume of tissue.

15. The method of claim 14, wherein rendering an image of the volume of tissue comprises rendering a merged image based on the distributions of the first and the second acoustomechanical parameters within the volume of tissue.

16. The method of claim 11, wherein rendering an image comprises rendering a three-dimensional image characterizing the volume of tissue.
